(19) United States
(12) Patent Application Publication
(10) Pub. No.: US 2021/0102247 A1
(43) Pub. Date: Apr. 8, 2021

(54) METHOD OF AMPLIFYING NUCLEIC ACIDS

(71) Applicant: Oxford University Innovation Limited, Oxford (GB)

(72) Inventors: Adam J. MEAD, Oxford (GB); Alba RODRIGUEZ-MEIRA, Oxford (GB)

(21) Appl. No.: 17/038,548

(22) Filed: Sep. 30, 2020

(30) Foreign Application Priority Data
Oct. 3, 2019 (GB) 1914266.0

(51) Int. Cl. C12Q 1/6858

(52) U.S. Cl. CPC C12Q 1/6858

(57) ABSTRACT

The present invention relates to a method of amplifying both genomic DNA and mRNA from a composition comprising one or more cells. The method comprises the step of treating a composition comprising one or more cells with a protease which is capable of being heat-inactivated at a temperature of less than 75° C., thus ensuring that any RNA in the composition is not degraded.

### CROSS-REFERENCE TO RELATED APPLICATION

This application claims priority to GB 1914266.0, filed Oct. 3, 2019, which is entirely incorporated herein by reference.

### FIELD OF THE INVENTION

The present invention relates to a method of amplifying both genomic DNA and mRNA from a composition comprising one or more cells. The method comprises the step of treating a composition comprising one or more cells with a protease which is capable of being heat-inactivated at a temperature of less than 75° C., thus ensuring that any RNA in the composition is not degraded.

### BACKGROUND

Tumours are composed of heterogeneous cell populations which can have different genetic and molecular properties. This diversity poses a major therapeutic challenge in cancer: the presence, in a single tumour, of many different genetic subclones, which might also be molecularly and functionally distinct, makes it difficult to effectively target all cancer cells in a particular patient, and this is a major cause of treatment failure.

Therefore, it is important to characterize the molecular and functional consequences of intra-tumoral heterogeneity, in order to develop better cancer treatments.

For many years, bulk sequencing techniques were used to analyse tumours as relatively uniform entities. Whilst these techniques identified some major genetic drivers of the disease, they did not provide the necessary resolution to resolve the phylogenetic tree of each tumour or to identify molecularly-distinct subpopulations of cells.

Single-cell genomic technologies allow the studying of cellular systems at extremely high resolution. Single-cell DNA sequencing techniques are used to reconstruct clonal hierarchies and to determine combinatorial patterns of mutations; and single-cell transcriptomic techniques have been used to identify molecularly distinct subpopulations of cells which correlate with disease progression (1) or which persist after treatment (1, 2).

Single-cell techniques are therefore ideally placed to resolve heterogeneity of cancerous tissues. However, the lack of technologies which correlate genetic and transcriptional readouts from the same single cell has limited the application of single-cell techniques to the study of tumours.

Linking genetic and whole transcriptome readouts would allow the determining of genotype-phenotype correlations (inferred from the transcriptome) unambiguously, and gain mechanistic insights into clonal evolution. However, the amplification of genetic material (genomic DNA or mRNA) from single cells is challenging, resulting in a high number of false-negative results. Therefore, the low molecular capture rate (commonly referred to as allelic dropout) of single-cell transcriptomic techniques has prevented the correlation of transcriptional and genetic readouts from the same cell (3, 4); likewise, single cell genomic techniques analysing mutations from the genomic DNA of single cells have not been compatible with parallel whole transcriptome amplification from the same single cell.

Several technologies have been developed for the analysis of mutations from the mRNA of single cells in parallel with whole transcriptome analysis. These technologies rely on the targeted amplification of mutated transcripts from single cells during retro-transcription or subsequently to cDNA amplification (5, 6). However, the lack of expression of the genes targeted in the majority of cells and the highly allelic-biased expression of mutated transcripts has resulted in mutation detection rates <5% for most of the genes targeted. Additionally, these methods are mostly suited for the analysis of mutations in the 3′-end of the transcripts, and they provide extremely low detection rates for mutations found many base-pairs away from the transcript end.

To circumvent this limitation, the inventors have developed a technology to simultaneously identify mutations from single cells and obtain whole transcriptome readouts. This technology relies on the targeted amplification of mutations from coding and genomic DNA, in parallel with oligo-dT-based whole transcriptome analysis.

By using parallel targeted amplification of mutations from coding and genomic DNA, the allelic dropout rate (i.e., the ability of detect both alleles from a gene in a single cell) of the method was reduced to less than 2%, which provides extremely high sensitivity of mutation detection to confidently reconstruct tumour clonal hierarchies. The amplification of mutations from genomic DNA allows the profiling of mutations which are not expressed in every single cell or expressed at very low levels, and circumvents allelic biases at the mRNA level (i.e., preferential expression of one of the alleles). The parallel amplification of mutations from coding DNA provides an independent readout of the mutational status of each single cell, increasing the resolution of the method. At the same time, whole transcriptome analysis was performed from the same single cell using an oligo-dT-based mRNA retro-transcription strategy (4).

Previous attempts to do this relied on the physical separation of genomic DNA and mRNA molecules from each single cell (7-9), which resulted in extremely high allelic dropout rates (estimated in >15% at the gene level and >30% at the allele level) likely due to the inevitably loss of genetic material. Another method relied on the parallel amplification of gDNA and mRNA with subsequent masking of coding regions (10), which made it impossible to distinguish mutational readouts from genomic DNA or coding DNA. The low confidence mutational information provided by all of these methods restricted their use to the study of cancerous tissues, and none of them have been widely applied to the resolution of intratumoral heterogeneity to date.

Alternatively, targeted amplification of transcripts and genomic DNA have previously provided high resolution mutational readouts from single cells. However, this approach was restricted to the analysis of a small subset (<96) of pre-selected transcripts, and therefore is not suitable for a discovery-type whole transcriptome analysis.

Lastly, a recently-developed method (CORTAD-seq, (11)) also allows parallel mutation and whole transcriptome analysis from the same single cell using. However, this protocol relies on a commercial formulation of lysis buffer (Polaris lysis plus reagent; Fluidigm) which achieves low quality whole transcriptome sequencing data.

It has now been found that the detection of specific mRNA and genomic DNA with parallel whole transcriptome analysis can be improved by modifying the previously-published protocols.

In particular it has been found that, if an additional lysis step is used utilising a protease, the release of genomic DNA from cells can be improved. However, the first proteases tested in this lysis step were found to interfere with the subsequent reverse-transcriptase and PCR-amplification steps; hence the protease had to be removed or inactivated before these latter steps could take place. Traditional proteases such as proteinase K require heat-treatment at very high temperatures (12): even when incubating at 95° C. for 10 minutes, some enzymatic activity of proteinase K remains. Such proteases could not therefore be used in this method due to the fact that mRNA is degraded at temperatures above 75° C.; hence alternative proteases or protease inhibitors had to be used. Following the testing of a number of proteases and incubation conditions, appropriate conditions were found which facilitate the inactivation of the protease and yet maintain the integrity of the mRNA.

### SUMMARY OF THE INVENTION

It is one object of the invention therefore to provide a method of amplifying genomic DNA and mRNA in parallel which allows for the improved detection of specific mRNA and genomic DNA amplicons.

In one embodiment, the invention provides a method of amplifying both genomic DNA and mRNA from a composition comprising one or more cells, the method comprising the steps:

(a) treating a composition comprising one or more cells with a protease to release genomic DNA and mRNA from the cells, wherein the protease is one which is capable of being heat-inactivated at a temperature of less than 75° C.;

(b) heat-treating the composition to inactivate the protease at a temperature of less than 75° C.;

(c) producing cDNA from the mRNA; and

(d) amplifying the genomic DNA and cDNA.

### DETAILS OF THE INVENTION

The composition comprises one or more cells. The cells may be prokaryotic or eukaryotic cells. Preferably, the cells are eukaryotic cells. Examples of eukaryotic cells include cells from animals, plants and fungi. Preferably, the eukaryotic cells are higher eukaryote cells or cells from multicellular organisms. The plants may be monocots or dicots.

In some embodiments, the eukaryotic cells are animal cells, preferably vertebrate cells, and more preferably mammalian cells. Preferably, the mammalian cells are from a human, monkey, mouse, rat, rabbit, guinea pig, sheep, horse, pig, cow, goat, dog or a cat. Most preferably, the mammalian cells are human cells.

In some embodiments, the cells are myeloid cells or stem cells (e.g. embryonic stem cells or hematopoietic stem cells). Preferably, the genomic DNA and mRNA are obtained from live cells.

Preferably, the composition comprises 1-2, 1-5, 1-10 or 1-100 cells. Most preferably, the composition comprises only one cell.

The composition preferably additionally comprises a Lysis Buffer. The Lysis Buffer is preferably an aqueous buffer. The Lysis Buffer may comprise (in addition to the protease) one or more of the following: (i) a detergent or surfactant (e.g. Triton X-100); (ii) an RNase inhibitor; (iii) oligonucleotides (e.g. oligo-dT primers); (iv) dNTPs; and (v) DNase/RNase-free water. Most preferably, the Lysis Buffer comprises all of (i)-(v).

In Step (a), the composition is treated with a protease. The purpose of this step is to release genomic DNA and mRNA from within the cells, and to make this genomic DNA and mRNA accessible for the subsequent cDNA-production and amplification steps.

A protease is an enzyme which catalyses proteolysis (i.e. the breakdown) of proteins into smaller polypeptides or single amino acids. Proteolysis is achieved by cleaving the peptide bonds within proteins. The protease may be a serine protease, cysteine protease, threonine protease, aspartic protease, glutamic protease, metalloprotease or an asparagine peptide lyase. Preferably, the protease is a serine protease (e.g. a subtilisin-related serine protease).

The protease is one which is capable of being heat-inactivated at a temperature of less than 75° C. This is to ensure that the mRNA is not degraded when the protease is in activated in the heat-treatment step of Step (b).

In some embodiments, the protease is one which is capable of being heat-inactivated at a temperature of less than 75° C., 74° C., 73° C., 72° C., 71° C. or 70° C. (e.g. after 15 minutes). In some embodiments, the protease is one which is capable of being heat-inactivated at a temperature of 70-75° C., 70-74° C., 70-73° C., 70-72° C. or 70-71° C. (e.g. after 15 minutes).

In some embodiments, the protease is one which is capable of being heat-inactivated at a temperature of 65-75° C., 65-74° C., 65-73° C., 65-72° C., 65-71° C. or 65-70° C. (e.g. after 15 minutes). In other embodiments, the protease is one which is capable of being heat-inactivated at a temperature of 55-65° C. or 60-70° C., e.g. 55-60° C., 60-65° C. or 65-70° C. (e.g. after 10 or 15 minutes). In other embodiments, the protease is one which is capable of being heat-inactivated at a temperature of 50-60° C., e.g. 50-55° C., preferably 52-58° C., more preferably 54-56° C. or about 55° C. (e.g. after 10 or 15 minutes).

In some embodiments, the protease is one which is capable of being heat-inactivated at one of the temperatures given above after 10-15 minutes or 12-20 minutes, e.g. after 10, 11, 12, 13, 14 or 15 minutes.

Most preferably, the protease is QIAGEN® Protease, catalogue no. 19155. This is a serine protease isolated from a recombinant Bacillus strain. QIAGEN® Protease is inactivated by incubation at 72° C. for 15 minutes. Another protease which could be used for the method is the thermo-labile Proteinase K, of New England Biolabs (Catalogue No. P8111). This is inactivated at 55° C. for 10 minutes. This enzyme is from the fungi Engyodontium album (Tritirachium album).

Step (a) is performed at a temperature of less than 75° C. in order to avoid degrading the mRNA. Preferably, the cells are treated in Step (a) at a temperature of 50° C. to 75° C., e.g. 50-55° C., 55-60° C., 60-65° C., 65-70° C. or 70-75° C., more preferably at a temperature of 70° C. to 72° C. Preferably, the cells are treated in Step (a) for 10 to 20 minutes, more preferably for 15 minutes.

The optimum pH for the protease may readily be selected by simple experimentation. The optimum pH may, for example, be pH 4-5, 5-6, 6-7, 7-8, 8-9, 9-10, 10-11 or 11-12. In some embodiments, the pH range may be 7.5 to 12.

The concentration of the protease should be such that genomic DNA and mRNA is released from the cells within the duration of the treating step. Preferably, the concentration of the protease in the composition is 2 E-05 to 4 E-05 Anson Units per microliter, more preferably 2.2 E-05 to 3.2 E-05, and even more preferably 2.5 E-05 to 2.9 E-05 or 2.6 E-05 to 2.8 E-05.

The duration, pH and temperature of the treating step, and the concentration of the protease, will be selected such that genomic DNA and mRNA is released from the cells, preferably a substantial amount (e.g. enough to sequence) of genomic DNA and mRNA is released from the cells.

Step (b) comprises heat-treating the composition to inactivate the protease at a temperature of less than 75° C. The purpose of this step is to ensure that the protease does not significantly degrade the enzymes used in the subsequent cDNA-production and amplification steps.

In some embodiments, the temperature of the heat-treating step is less than 75° C., 74° C., 73° C., 72° C., 71° C. or 70° C. In some embodiments, the temperature is 70-75° C., 70-74° C., 70-73° C., 70-72° C. or 70-71° C. Preferably, the temperature is above 65° C.

In some embodiments, the temperature is 65-75° C., 65-74° C., 65-73° C., 65-72° C., 65-71° C. or 65-70° C. Preferably, the temperature is 71-73° C., more preferably about 72° C. In other embodiments, the temperature is 55-70° C., e.g. 55-60° C., 60-65° C. or 65-70° C. In other embodiments, the temperature is 50-60° C., e.g. 50-55° C., preferably 52-58° C., more preferably 54-56° C. or about 55° C.

Preferably, the heat-treating is at one of the temperatures given above for 10-15, 12-15 or 12-20 minutes, e.g. 13-15, 14-15 or 15 minutes. Preferably, the heat-treating is at 71-73° C. for 14-16 minutes. In other preferred embodiments, the heat treating is at 55° C. for 9-11 minutes.

Preferably, the protease has lost at least 80%, 85%, 90%, 95%, 99% or 100% of its activity after the heat-treatment step (Step (b)) compared to its activity before the heat-treatment step. The degree of protease activity may be tested by using a fluorometric assay of an appropriate digestion product, for example. Additionally, quantifying the cDNA yield obtained may be used as an indirect measure of proteinase activity.

The heat in the heat-treatment step may be applied to the composition by any suitable means, e.g. in a PCR thermocycler.

Step (c) comprises producing cDNA from the mRNA. This step will generally comprise the use of mRNA primers (including a template switching oligonucleotide), RNase inhibitor and reverse transcriptase. Protocols to perform this step are well known in the art. The mRNA primers may be target-specific primers.

Step (c) may also comprise the step of tagging one or both ends of the cDNA molecules with adaptor sequences.

Step (d) comprises amplifying the genomic DNA and the cDNA which was produced in Step (c). This step will generally comprise the use of DNA primers and PCR amplification. Protocols to perform this step are well known in the art.

In at least part of the amplification step, the genomic DNA and cDNA are amplified in parallel, preferably together, using independent target-specific primers. Preferably, the genomic DNA and cDNA are amplified in the same reaction, but using different primers for each type of molecule.

Preferably, Step (d) comprises:
(d)(i) amplification of genomic DNA and cDNA using target-specific primers; and
(d)(ii) amplification of the whole cDNA using non-specific or specific primers.

Steps (d)(i) and (d)(ii) are preferably carried out separately.

The primers used in Step (d)(ii) may alternatively be primers which bind to an adaptor sequence which has been tagged to the cDNA molecules during Step (c). In this case, they are “specific” primers.

Optionally, the method of the invention additionally comprises the step:
(e) sequencing all or part of the amplified genomic DNA and/or amplified cDNA.

Preferably, Step (e) comprises the step:
(e)(i) using NGS to sequence the amplified genomic DNA and cDNA produced in Step (d)(i); and/or
(e)(ii) sequencing the whole cDNA produced in Step (d)(ii).

(“NGS” refers to Next Generation Sequencing.)

The disclosure of each reference set forth herein is specifically incorporated herein by reference in its entirety.

### BRIEF DESCRIPTION OF THE FIGURES

FIG. 1: Comparison of different strategies for proteinase-based lysis of single cells. (A) Comparison of cDNA yield obtained from single cells obtained after lysis with a Proteinase K (Catalogue No. P8107S) and subsequent inhibition with a Proteinase K Inhibitor (Calbiochem, Catalogue No. 539470-10MG) or lysis with Qiagen Protease (Catalogue No. 19155) and subsequent heat inactivation at 72° C. SMART-seq plus condition (Rodriguez-Meira et al.), which did not include proteinase in the lysis step, was used as a control condition. Proteinase K or Qiagen Protease were added during the lysis step and addition of inhibitor or heat inactivation was performed prior retro-transcription. (B) Comparison of cDNA yield obtained from single cells after lysis with a Proteinase K (Catalogue No. P8107S) or lysis with Qiagen Protease (Catalogue No. 19155) and subsequent heat-inactivation of the proteinases at 95° C. SMART-seq plus condition, not including a proteinase-based digestion, was used for comparison. Proteinase K or Qiagen Protease were added after the retrotranscription step and heat inactivation was performed prior PCR amplification of cDNA molecules.

FIG. 2: Comparison of different heat inactivation lengths using Qiagen Protease. Sequencing statistics from single cells lysed using standard SMART-seq plus detergent-based conditions (Condition 1) or including Qiagen Protease in the lysis buffer, subsequently heat-inactivated for 10 minutes (Condition 2) or 15 minutes (Condition 3). 2.7 E-05 Anson Units per microliter were used for Conditions 2 and 3. The number of reads in genes indicates the efficiency of each condition in detecting cDNA molecules mapping to known transcripts versus background sequencing noise. The number of genes detected per cell indicates the molecular capture rate of the method. The library bias was calculated as the mean expression values (in RPKM; Reads Per Kilobase per Million reads) of the top 10% highly expressed genes versus the average RPKM values of all of the genes detected, and indicates the bias of the method towards detecting highly expressed genes. “fc” indicates fold-change deviation from the mean of the control group (Condition 1).

FIG. 3: Comparison of different Qiagen Protease concentrations in the lysis buffer. Sequencing statistics from single cells lysed using standard SMART-seq plus detergent-based conditions (Condition 1) or including 1.35 E-05 Anson Units per microliter (Condition 2), 2.7 E-05 Anson Units per microliter (Condition 3) or 5.4 E-05 Anson Units per microliter (Condition 4) of Qiagen Protease in the lysis buffer, with subsequent heat-inactivation of the protease for 15 minutes. The number of reads in genes indicates the efficiency of each condition in detecting cDNA molecules mapping to known transcripts versus background sequencing noise. The number of genes detected per cell indicates the molecular capture rate of the method. The library bias was calculated as the mean expression values (in RPKM; Reads Per Kilobase per Million reads) of the top 10% highly expressed genes versus the average RPKM values of all of the genes detected, and indicates the bias of the method towards detecting highly expressed genes. “fc” indicates fold-change deviation from the mean of the control group (Condition 1).

FIG. 4: Combined protease digestion and targeted amplification of genomic DNA regions efficiently detected mutations in single cells. (A) Frequency of bi-allelic detection (detection of both alleles) of ten heterozygous point mutations and short indels in clonal cell lines. Addition of protease and primers (Condition 3) increased the detection of mutations to 93%, from 2% average detection in control condition (SMART-seq plus, not including primers or protease; Condition 1) and from 16% average detection when only using target-specific primers but not including protease digestion (Condition 2).

FIG. 5: Combined protease digestion and concomitant targeted amplification of genomic DNA and mRNA increases the efficiency of detection of mutations in single cells. (A) Frequency of bi-allelic detection (detection of both alleles) of ten heterozygous point mutations and short indels in clonal cell lines when combining mutational readouts from targeted gDNA and targeted mRNA from single cells. Addition of protease and primers (Condition 3) increased the detection of mutations to 96.4%, from 27.8% average detection in control condition (SMART-seq plus, not including primers or protease; Condition 1); and from 57.4% average detection when only using target-specific primers but not including protease digestion (Condition 2).

### EXAMPLES

The present invention is further illustrated by the following Examples, in which parts and percentages are by weight and degrees are Celsius, unless otherwise stated. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various usages and conditions. Thus, various modifications of the invention in addition to those shown and described herein will be apparent to those skilled in the art from the foregoing description. Such modifications are also intended to fall within the scope of the appended claims.

### Example 1: Testing and Selection of Proteases

Several proteinases and lysis conditions were tested to determine the optimal cell-lysis conditions. cDNA yield from single cells was used as a measurement of efficient retro-transcription and cDNA amplification, compared to a control condition.

Two different proteases were tested for the efficient retro-transcription and amplification of cDNA: proteinase K from New England Biolabs (Catalogue No. P8107S) and Qiagen Protease (Catalogue No. 19155). Two different conditions were also tested: addition of the proteinases in the lysis buffer, and subsequent heat-inactivation before performing retro-transcription; and addition of the proteinase after the retro-transcription step, with subsequent inactivation prior performing PCR amplification.

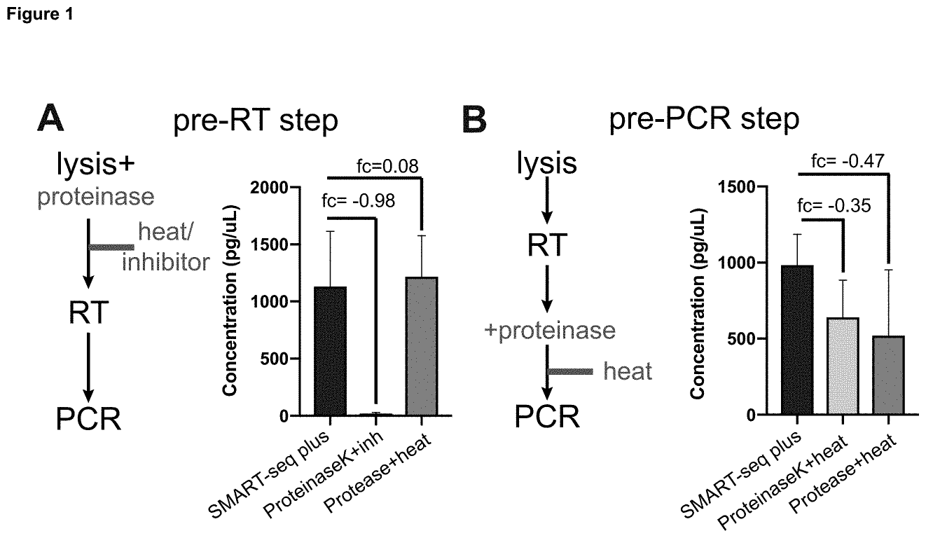

In the first instance, addition of the proteinase in the lysis buffer was tested. Proteinase K can only be efficiently inactivated by heat at 95° C., at which temperature the mRNA is degraded. Therefore, Proteinase K was inactivated by the addition of a proteinase inhibitor (Proteinase K inhibitor, Calbiochem, Catalogue No. 539470-10MG). Digestion with Proteinase K and proteinase inhibitor resulted in a complete inhibition of cDNA amplification as compared to control conditions (98% reduction in cDNA yield). In contrast, digestion with Qiagen Protease (Catalogue No. 19155) and subsequent heat-inactivation at 72° C. did not affect cDNA yield, indicating that protease was efficiently inactivated by heat (FIG. 1A).

In the second instance, the addition of proteinase after the retro-transcription and prior to performing a PCR amplification step was tested. Proteinase K from New England Biolabs (Catalogue No. P8107S) and Qiagen Protease (Catalogue No. 19155) were tested. Both enzymes were added to a composition of one single cell in a PCR plate and were incubated at 56° C. for 10 minutes and heat-inactivated at 95° C. for 20 minutes. The addition of proteinase in this step resulted in a 35 to 47% reduction in cDNA yield (FIG. 1B).

### Example 2: Optimisation of Duration of Heat-Inactivation

Two different lengths of heat inactivation at 72° C. were tested in single human haematopoietic stem and progenitor cells, to determine the optimal duration of heat inactivation of the Qiagen Protease.

cDNA libraries from single cells were prepared using the commercially-available Nextera XT Library Preparation Kit (Illumina, Catalogue No. FC-131-1096) and sequenced on a NextSeq instrument (IIlumina). Reads were aligned to the human genome using STAR and reads mapping to each transcript were quantified using featureCounts. Then, several metrics were calculated for single cells from each condition: the percentage of reads mapping to known transcripts, which determines the efficiency of the lysis, retro-transcription and PCR steps; the number of genes detected per cell, which determines the molecular capture rate of each condition; and the library bias, which indicates the bias of each condition towards detecting highly expressed genes.

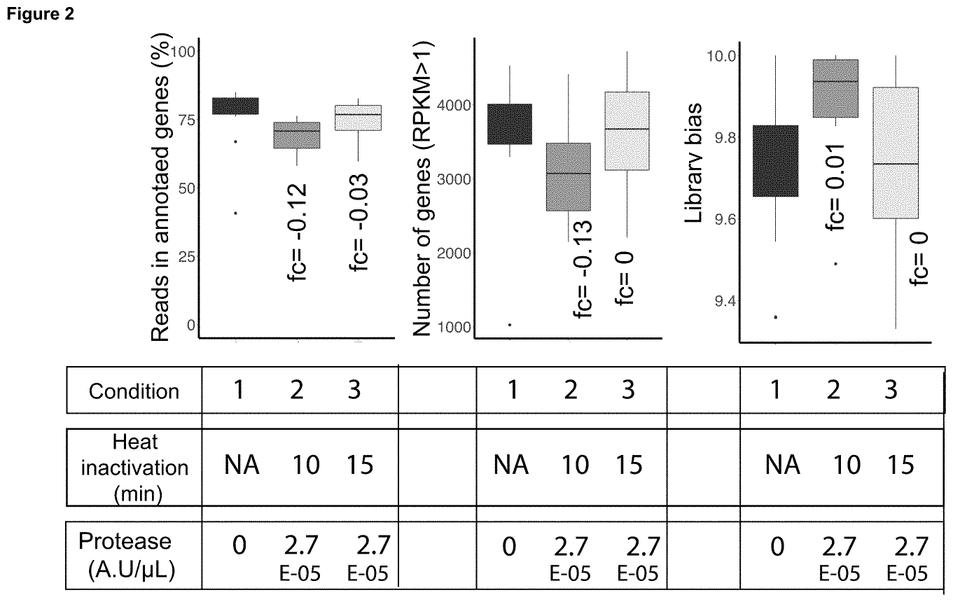

Qiagen Protease was added to the lysis buffer and heat inactivated for 10 minutes or 15 minutes, and sequencing metrics were compared to a control condition which did not include Qiagen Protease. Inactivating Qiagen Protease for 10 minutes led to a 12% reduction in reads mapping in known transcripts, 13% reduction in genes detected per cell and an increase in library bias (FIG. 2). Inactivation of Qiagen Protease for 15 minutes achieved sequencing metrics comparable to control conditions (3% decrease in reads mapping in known transcripts, and no differences in number of genes detected per cell or library bias; FIG. 2). This indicated that applying 72° C. for 15 minutes efficiently inactivated Qiagen Protease and led to degeneration of good quality cDNA libraries from single cells.

### Example 3: Optimisation of Protease Concentration

Three different Qiagen Protease concentrations (1.54 E-05, 2.7 E-05 and 5.4 E-05 Anson Units per microliter) were tested in single human haematopoietic stem and progenitor cells, to determine the optimal concentration of Qiagen Protease in the lysis buffer. Qiagen protease was added to the lysis buffer and heat inactivated at 72° C. for 15 minutes prior retro-transcription.

cDNA libraries from single cells were prepared using the commercially available Nextera XT Library Preparation Kit (Illumina, Catalogue No. FC-131-1096) and sequenced on a NextSeq instrument. Reads were aligned to the human genome using STAR and reads mapping to each transcript were quantified using featureCounts. Then, several metrics were calculated for single cells from each condition: the percentage of reads mapping to known transcripts, which determines the efficiency of the lysis, retro-transcription and PCR steps; the number of genes detected per cell, which determines the molecular capture rate of each condition; and the library bias, which indicates the bias of each condition towards detecting highly expressed genes.

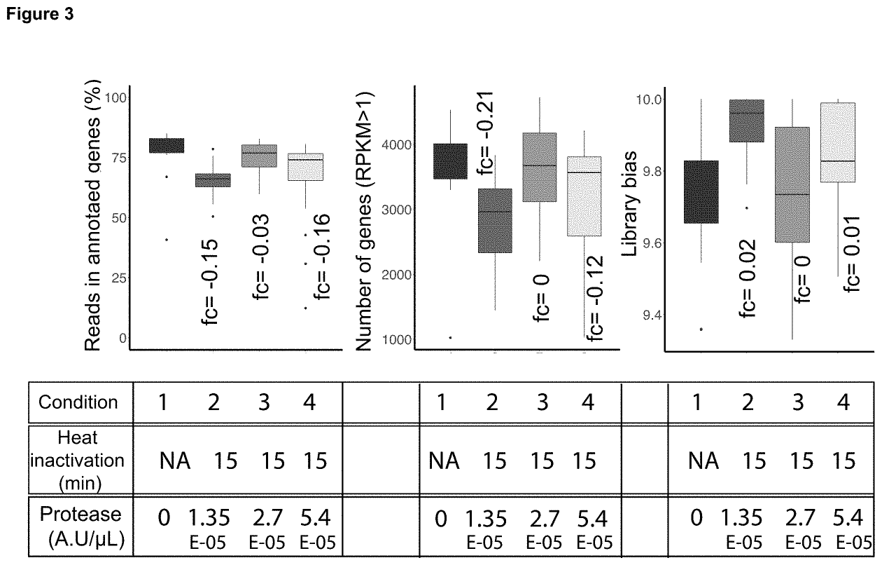

Qiagen Protease was added to the lysis buffer in different concentrations and heat inactivated for 15 minutes; then sequencing metrics were compared to a control condition which did not include Qiagen Protease. The addition of 1.54 E-05 or 5.4 E-05 Anson Units per microliter led to a reduction in the reads mapping in transcripts (15% and 16% respectively), a reduction in the number of genes detected per cell (21% and 12% respectively) and an increase in library bias (FIG. 3). Addition of 2.7 E-05 Anson Units per microliter achieved comparable sequencing metrics to control condition (3% reduction in reads mapping in known transcripts and no difference in number of genes detected per cell or library bias). This indicated that addition of 2.7 E-05 Anson Units per microliter was the optimal concentration of Qiagen Protease that led to degeneration of good quality cDNA libraries from single cells.

### Example 4: Method of Detecting Mutations from Genomic DNA and mRNA from Single Cells in Parallel with Whole Transcriptome Amplification

To efficiently amplify mutations from genomic DNA and mRNA from single cells, cells were first lysed with a lysis buffer composed of Triton X-100, RNase inhibitor, oligodT primer, dNTPs (Conditions 1 and 2) and, in the case of Condition 3, 2.7 E-05 Anson Units per microliter of Qiagen Protease were also added. Then, samples were incubated at 72° C. for 15 minutes in a thermocycler, and a retrotranscription mix was subsequently added. This retrotranscription mix contained a MMLV-derived retrotranscriptase (SMARTScribe, Clontech, Catalogue No. 639538), a template switching oligonucleotide attached to an adaptor sequence and RNAse inhibitor for SMART-seq plus condition (Control, Condition 1). Condition 2 and Condition 3 (TARGET-seq) included mRNA-specific primers targeting mRNA molecules of interest containing known heterozygous mutations in RUNX1, NOTCH1, TP53, PTEN, JAK2, TET2, U2AF1, NRAS, and two intronic SNPs in chromosome 9 (chr9-SNP1, chr9-SNP2). Then, PCR was performed using SeqAMP DNA Polymerase (Clontech, Catalogue No. 638504) and a primer binding to the adaptor sequence contained within the oligodT and template switching oligonucleotide for SMART-seq plus condition. Primers targeting specific cDNA molecules and primers targeting genomic DNA amplicons spanning the mutations of interest were also added to the mix for Conditions 2 and 3. Then, targeted libraries for each mutation were prepared and sequenced by Next Generation Sequencing (IIlumina). Variant calling was performed in each single cell and the percentage of cells in which a particular mutation was detected as heterozygous was quantified for each method.

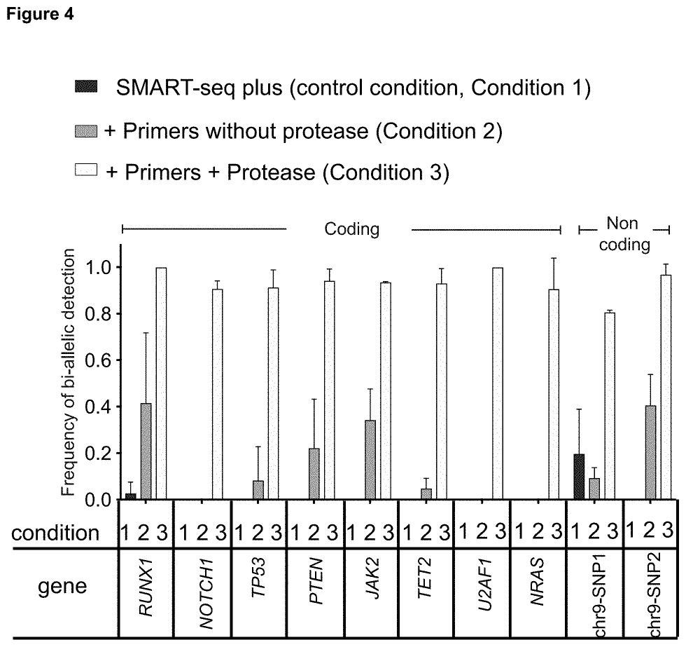

Analysis of mutations from genomic DNA amplicons in Condition 3 (TARGET-seq method) achieved a mean frequency of detection of 93% (FIG. 4), as compared to 2% average detection in the Control condition (SMART-seq plus, not including primers of protease; Condition 1) and from 16% average detection when only using target-specific primers but not including protease digestion (Condition 2).

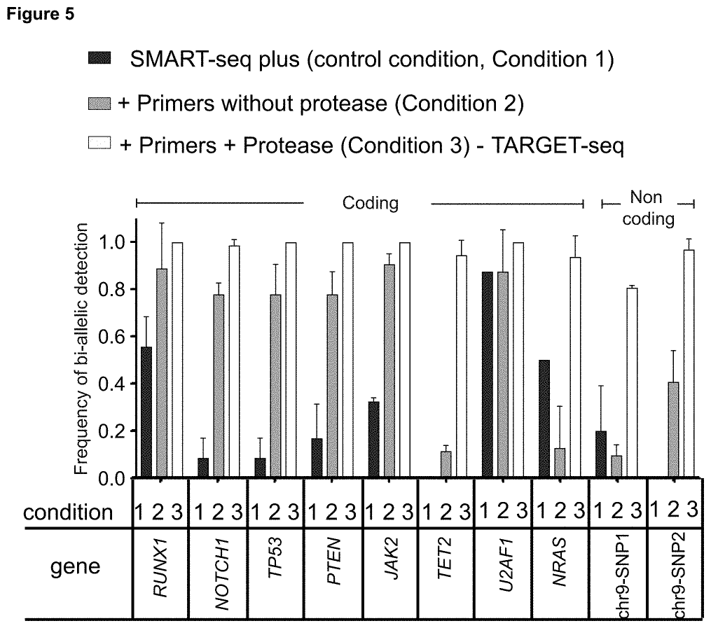

Concomitant analysis of mutations from genomic DNA and mRNA amplicons increased the accuracy of mutation detection in single cells. Condition 3 achieved a mean frequency of detection of 96.4% (FIG. 5), as compared to 27.8% mean detection in the Control condition (SMART-seq plus, not including primers of protease; Condition 1) and from 57.4% average detection when only using target-specific primers but not including protease digestion (Condition 2).

### REFERENCES

1. Giustacchini A, Thongjuea S, Barkas N, Woll P S, Povinelli B J, Booth C A G, et al. Single-cell transcriptomics uncovers distinct molecular signatures of stem cells in chronic myeloid leukemia. Nat Med. 2017; 23(6):692-702.

2. Tirosh I, Izar B, Prakadan S M, Wadsworth M H, 2nd, Treacy D, Trombetta J J, et al. Dissecting the multicellular ecosystem of metastatic melanoma by single-cell RNA-seq. Science. 2016; 352(6282):189-96.

3. Tirosh I, Venteicher A S, Hebert C, Escalante L E, Patel A P, Yizhak K, et al. Single-cell RNA-seq supports a developmental hierarchy in human oligodendroglioma. Nature. 2016; 539(7628):309-13.

4. Rodriguez-Meira A, Buck G, Clark S A, Povinelli B J, Alcolea V, Louka E, et al. Unravelling Intratumoral Heterogeneity through High-Sensitivity Single-Cell Mutational Analysis and Parallel RNA Sequencing. Mol Cell. 2019; 73(6):1292-305 e8.

5. Nam A S, Kim K T, Chaligne R, Izzo F, Ang C, Taylor J, et al. Somatic mutations and cell identity linked by Genotyping of Transcriptomes. Nature. 2019.

6. van Galen P, Hovestadt V, Wadsworth Ii M H, Hughes T K, Griffin G K, Battaglia S, et al. Single-Cell RNA-Seq Reveals AML Hierarchies Relevant to Disease Progression and Immunity. Cell. 2019; 176(6):1265-81 e24.

7. Macaulay I C, Haerty W, Kumar P, Li Y I, Hu T X, Teng M J, et al. G&T-seq: parallel sequencing of single-cell genomes and transcriptomes. Nat Methods. 2015; 12(6):519-22.

8. Hou Y, Guo H, Cao C, Li X, Hu B, Zhu P, et al. Single-cell triple omics sequencing reveals genetic, epigenetic, and transcriptomic heterogeneity in hepatocellular carcinomas. Cell Research. 2016; 26(3):304-19.

9. Han K Y, Kim K T, Joung J G, Son D S, Kim Y J, Jo A, et al. SIDR: simultaneous isolation and parallel sequencing of genomic DNA and total RNA from single cells. Genome Res. 2018; 28(1):75-87.

10. Dey S S, Kester L, Spanjaard B, Bienko M, van Oudenaarden A. Integrated genome and transcriptome sequencing of the same cell. Nat Biotechnol. 2015; 33(3):285-9.

11. Kong S L, Li H, Tai J A, Courtois E T, Poh H M, Lau D P, et al. Concurrent Single-Cell RNA and Targeted DNA Sequencing on an Automated Platform for Comeasurement of Genomic and Transcriptomic Signatures. Clin Chem. 2019; 65(2):272-81.

12. Bajorath J, Hinrichs W, Saenger W. The enzymatic activity of proteinase K is controlled by calcium. European Journal of Biochemistry. 1988; 176(2):441-7.

1. A method of amplifying both genomic DNA and mRNA from a composition comprising one or more cells, the method comprising the steps:
(a) treating a composition comprising one or more cells with a protease to release genomic DNA and mRNA from the cells, wherein the protease is one which is capable of being heat-inactivated at a temperature of less than 75° C.;
(b) heat-treating the composition to inactivate the protease at a temperature of less than 75° C.;
(c) producing cDNA from the mRNA; and
(d) amplifying the genomic DNA and cDNA.

2. The method as claimed in claim 1, wherein the cells are eukaryotic cells, or mammalian cells, or human cells.

3. The method as claimed in claim 1, wherein the cells are myeloid cells or stem cells.

4. The method as claimed in claim 1, wherein the composition comprises only one cell.

5. The method as claimed in claim 1, wherein the protease is a serine protease.

6. The method as claimed in claim 1, wherein the protease is a bacterial or fungal protease, or a Bacillus or Engydontium protease.

7. The method as claimed in claim 1, wherein the protease is:
(i) capable of being heat-inactivated at a temperature of 70-75° C. and the heat-treating is at a temperature of 70-75° C.; or
(ii) capable of being heat-inactivated at a temperature of 50-60° C. and the heat-treating is at a temperature of 50-60° C.

8. The method as claimed in claim 1, wherein in Step (d), the genomic DNA and cDNA are amplified in parallel.

9. The method as claimed in claim 1, wherein Step (d) comprises the steps:
(d)(i) amplification of the genomic DNA and the cDNA using target-specific primers; and
(d)(ii) amplification of the whole cDNA using non-specific or specific primers.

10. The method as claimed in claim 1, wherein the method additionally comprises the step:
(e) sequencing all or part of the amplified genomic DNA and/or the amplified cDNA.

11. The method as claimed in claim 9, wherein the method additionally comprises the step:
(e)(i) using NGS to sequence the amplified genomic DNA and cDNA produced in Step (d)(i); and/or
(e)(ii) sequencing the whole cDNA produced in Step (d)(ii).

12. The method as claimed in claim 10,
wherein the composition comprises a single cell;
Step (d) comprises amplifying the genomic DNA and cDNA using target-specific primers which span a site of interest; and
Step (e) comprises using NGS to sequence the amplified DNA and cDNA produced in Step (d);
in order to amplify and sequence amplicons spanning a target site of interest.
